(19) United States
(12) Patent Application Publication
(10) Pub. No.: US 2015/0305828 A1
(43) Pub. Date: Oct. 29, 2015

(54) APPARATUS FOR ADJUSTING A ROBOTIC SURGERY PLAN

(71) Applicant: CUREXO, Inc, Seoul (KR)

(72) Inventors: Young-bae PARK, Cheongju-si (KR); Chang-hun SONG, Goyang-si (KR); Jae-jun LEE, Suwon-si (KR)

(21) Appl. No.: 14/697,840

(22) Filed: Apr. 28, 2015

(30) Foreign Application Priority Data
Apr. 29, 2014 (KR) 10-2014-0051464

(51) Int. Cl. A61B 19/00; G06T 19/00; G09G 5/377; G06F 3/01

(52) U.S. Cl. CPC A61B 19/56; G06F 3/011; G06T 19/006; G09G 5/377; A61B 2019/562; A61B 2019/566; A61B 2019/568; G06T 2207/30004; G09G 2340/12

(57) ABSTRACT

Disclosed is an apparatus for adjusting a robotic surgery plan and a method thereof. The apparatus according to the present invention comprises a surgery information storage unit storing an examined first image associated with an inputted robotic surgery plan and a target bone of surgery, a scene image obtaining unit obtaining a second image associated with a diseased part in real time in surgery room, an image registration unit matching coordinates of the examined first image with coordinates of the second image associated with the diseased part, a user interface displaying the examined first image and the second image associated with the diseased part, and a surgery control unit controlling the user interface so that the user interface displays the examined first image to be superimposed on the second image associated with the diseased part, which is inputted in real time.

### BACKGROUND OF THE INVENTION

1. Field of the Invention

The present invention relates to a robotic surgery. More specifically, the present invention relates to an apparatus for adjusting a robotic surgery plan.

2. Description of the Related Art

The deepening of a low birthrate and an aging phenomenon is acting as a catalyst in developing robotic industry. As the need for smart robots working instead of people increases, the worldwide robot market is rapidly expanding. The robots can be utilized in various fields, including operations in biologically dangerous regions like the scene of a fire, the reconnaissance in battlefield, and the lengthy surgery, etc.

Among those robots, medical robots have been being developed focusing most on user convenience. The main principles in developing medical robots are to provide convenience in using to doctors, to provide no inconvenience to patients, to minimize invasions of patients, to minimize pains of patients, etc. The medical robot technology is a technic field combining BT (Bio-Tech), NT (Nano-Tech), RT (Robot-Tech), and MT (Medical-Tech).

Although the orthopedic surgery using a robot enables elaborate and precise bone cutting, it has problems to increase surgery time and cost caused by using robot equipment. In addition, when the orthopedic surgery is performed using a robot, there is a need to make a decision about how the robot cuts bones. If the decision is made before surgery, there are problems to take more time in addition to surgery, and to have difficulty to apply any anatomical information discovered during surgery. On the other hand, if the decision is made during surgery, there is a problem the surgery time is increased.

Although the direction of surgery can be planned before the beginning of surgery based on medical images and status of a patient, the surgery plan should be able to be modified during surgery because a surgeon might modify osteoplastic goals based on real anatomical information and lesions of the patient. Such modification of surgical plan should be made as soon as possible and securely. However, among the common systems or known systems, there is little or no product considering such matters. The known orthopedic robot system only allows planning surgery before surgery, and adjusting the plan a little bit during surgery, using identical user interface.

For instance, ROBODOC (Curexo Technology Corp, USA, California) provides a method to decide a position of an artificial joint based on preoperative CT bone images of a patient before surgery, and cut the bone in order to insert the artificial joint into the predetermined position during surgery (U.S. Pat. No. 6,430,434 etc.). However, according to the method, it has difficulty in modifying the position of implants based on intraoperative lesions or in modifying the approach direction of the robot during surgery.

In addition, MAKOplasty (Mako surgical, USA, Florida) allows deciding a position of an artificial joint just before surgery in surgery room, and modifying the plan of surgery after incising a diseased part in surgery room. Furthermore, it has the advantage that the approach direction of the robot is decided by doctor, not by robot. The doctor decides the approach direction of the robot by pulling the robot with his hands. However, according to the MAKOplasty as well, the surgery plan has to be decided based on CT images of bones. Because the CT images show only the shape of bones, to modify the surgery plan based on lesions of the patient not showed on the CT images, after observing lesions with eyes, while watching the CT images on display of a robot controller, the doctor should modify the surgery plan based on the status of lesions. Therefore, the MAKOplasty also has difficulty in modifying the surgery plan as ROBODOC.

Therefore, there is a need for a robot system, which can apply modifications of the surgery plan during surgery properly.

### SUMMARY OF THE INVENTION

It is an object of the present invention, which is to overcome aforementioned problems, to provide an apparatus enabling to adjust a robotic surgery plan actively and flexibly.

In accordance with one aspect of the present invention, there is provided an apparatus for adjusting a robotic surgery plan, including a surgery information storage unit storing an examined first image associated with an inputted robotic surgery plan and a target bone of surgery, a scene image obtaining unit obtaining a second image associated with a diseased part in real time in surgery room, an image registration unit matching coordinates of the examined first image with coordinates of the second image associated with the diseased part, a user interface displaying the examined first image and the second image associated with the diseased part, and a surgery control unit controlling the user interface so that the user interface displays the examined first image to be superimposed on the second image associated with the diseased part, which is inputted in real time.

The surgery information storage unit could further store phased cutting options of the robotic surgery plan and related images thereof.

The surgery control unit could provide at least one image associated with the cutting options that is applicable to a corresponding surgery step, according to request for modifying the surgery plan inputted via the user interface.

The surgery control unit could control the user interface so that at least one image associated with the cutting options is superimposed on the second image associated with the diseased part, and also displayed to be distinguishable from the second image associated with the diseased part.

The scene image obtaining unit could include an optical camera and a mechanical arm that is attached to the optical camera and supports movements of the optical camera.

The scene image obtaining unit and the user interface could be attached to each other to be moveable together.

The surgery control unit could control the user interface so that the user interface displays outlines of the first image to be superimposed on the second image associated with the diseased part.

The surgery control unit could display the outlines of the first image to be superimposed on the second image associated with the diseased part using augmented reality technology.

The surgery control unit could modify the robotic surgery plan based on a selected cutting option.

The apparatus for adjusting a robotic surgery plan further include a cutting robot processing a target bone of surgery according to the modified robotic surgery plan inputted from the surgery control unit.

The apparatus for adjusting a robotic surgery plan mentioned above can respond actively and promptly to various requests of modifications of plan during robotic surgery performed according to pre-inputted sequence.

### BRIEF DESCRIPTION OF THE DRAWINGS

FIG. 1 is a schematic diagram of an apparatus for adjusting a robotic surgery plan according to the present invention.

FIG. 2 illustrates an example of a scene image obtaining unit and a user interface according to the present invention.

FIG. 3 illustrates another example of the scene image obtaining unit and the user interface according to the present invention.

FIG. 4 illustrates an example of a screen of cutting options provided during surgery by the apparatus for adjusting a robotic surgery plan according to the present invention.

FIG. 5 is a flowchart that depicts a method for adjusting a robotic surgery plan according to the present invention.

FIG. 6 illustrates an example of a screen of the user interface in which the present invention can be applied.

### DETAILED DESCRIPTION OF THE INVENTION

Hereinafter, exemplary embodiments of the present invention will be described in detail. However, the present invention is not limited to the exemplary embodiments disclosed below, but can be implemented in various forms. The following exemplary embodiments are described in order to enable those of ordinary skill in the art to embody and practice the invention.

It will be understood that, although the terms first, second, etc. may be used herein to describe various elements, these elements should not be limited by these terms. These terms are only used to distinguish one element from another. For example, a first element could be termed as a second element, and similarly, a second element could be termed as a first element, without departing from the scope of the present invention. The term and/or used herein includes any or all combinations of one or more of the associated listed items.

It will be understood that when an element is referred to as being connected or coupled to another element, it can be directly connected or coupled to the other element or intervening elements may be present.

The terminology used herein is for the purpose of describing particular embodiments only and is not intended to be limiting of the invention. As used herein, the singular forms a, an and the are intended to include the plural forms as well, unless the context clearly indicates otherwise. It will be further understood that the terms comprises, comprising, includes and/or including, when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meanings as commonly understood by one of ordinary skill in the art to which this invention belongs. It will be further understood that terms, such as those defined in commonly uses dictionaries, should be interpreted as having a meaning that is consistent with their meaning in the context of the relevant art and will not be interpreted in an idealized or overly formal sense unless expressly so defined here.

Hereinafter, preferred embodiments of the present invention will be described in detail with the accompanying drawings. In the following description, the same reference numerals denote the same elements to facilitate the overall understanding, and repeated description thereof will be omitted.

The human body comprises bones, skins, muscles, etc. In the specification, the term “tissues” means a part of tissues of body, and the term “soft tissues” means tissues such as skins and muscles, etc. except bones in the body tissues. The term “images” used herein includes static images and moving images.

The present invention considers difficult problems coming up when a robotic surgery plan needs to be modified.

For example, when the surgery plan modifies, there may be a case to modify positions and angles of bones that are to be cut. On the other hand, there may be a case to modify approach directions or angles of robot without modifying the positions or angles of bones to be cut. Furthermore, because of difficulty in accessing to bones due to soft tissues, there also may be a case to finish with hands instead of the robot.

Therefore, the present invention displays an image associated with a processing plan intended to modify to be superimposed on an image of real diseased part, during surgery. Thus, the present invention can support surgeons' judgements effectively in surgery room, where swift decisions are required. For this, the present invention provides images of options for modifying the surgery plan using augmented reality technology. Accordingly, the present invention enables surgeons to modify the surgery plan swiftly and securely.

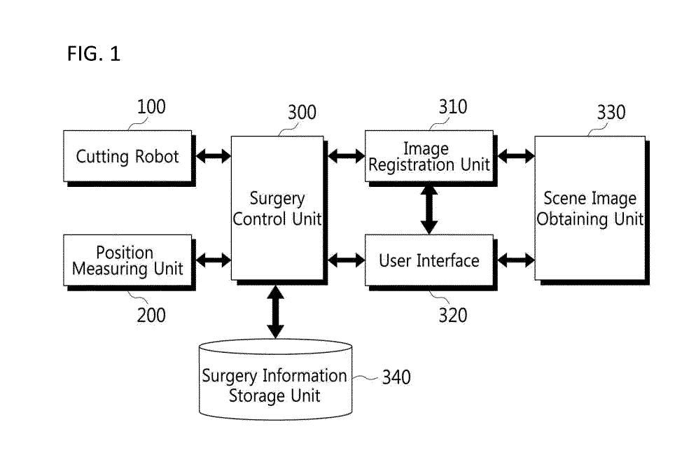

FIG. 1 is a schematic diagram of an apparatus for adjusting a robotic surgery plan according to the present invention.

Hereafter, the elements according to the present invention, which will be described by referring to FIG. 1, are those elements defined by functional classification, not by physical classification. The elements according to the present invention could be defined by functions performed by each of the elements. Each of the elements could be implemented as hardware and/or program codes performing each function and processing units. They also could be implemented so that the functions of two or more elements are included in one element. Therefore, it needs to be noted that the names of elements, given in following embodiments, are not for distinguishing elements physically, for representing main function performed by each element. Furthermore, it needs to be noted that the spirit of the present invention is not be limited by the names of elements.

As illustrated in FIG. 1, the apparatus for adjusting a robotic surgery plan according to the present invention comprises a cutting robot 100 to which surgery equipment for cutting bones by using an orthopedic surgery robot is attached, a position measuring unit 200 measuring the position of bones, a surgery control unit 300 finding the position of bones and determining cutting paths, a scene image obtaining unit 330 (for example, cameras etc.), an image registration unit 310, a user interface 320, and a surgery information storage unit 340.

The position measuring unit 200 measures the position of bones exposed outwards by incising skins and skin tissues in surgery. Digitizers, infrared units, laser units, etc. could be used for measuring the position of bones.

The surgery control unit 300 determines the real position of bones by matching three-dimensional shape images of bones, which is obtained by computerized tomography equipment, etc. before surgery, with three-dimensional position data obtained by the position measuring unit 200. Accordingly, the cutting robot 100 can determine exact cutting positions, and cutting paths.

Herein, the step of matching the three-dimensional shape images of bones, which is obtained by computerized tomography equipment, etc. before surgery, with the three-dimensional position data obtained by the position measuring unit 200 is referred to as registration.

In robotic surgery, the position registration is a step to calculate preferred surgery position based on the anatomical position of bones measured by an anatomical position finder and a surgery robot. Although, there are various methods for registration, the most representative registration method is explained hereafter.

In robotic surgery, the coordinate systems are classified into a reference coordinate system {F}, a robot coordinate system {R} about paths programmed in robot, and a bone coordinate system {B} about bones of a patient in real surgery. For registration, at first, convert the robot coordinate system {R} into a relative coordinate system relative to the reference coordinate system {F}, and convert the bone coordinate system {B} into the relative coordinate system relative to the reference coordinate system {F}. Thereby the robot coordinate system {R} and the bone coordinate system {B} are converted relative to the same reference coordinate system {F}. After that, calculate transformation matrix T of the converted robot coordinate system {R} and the converted bone coordinate system {B}, and apply the transformation matrix T into the converted robot coordinate system {R}. Thus, a processing path of robot can be applied appropriately according to the real position of bones.

As the registration method for calculating the transformation matrix T, there are pin-basis registration, image-basis registration, etc.

According to the pin-basis registration method, before surgery, with pins inserted from a diseased part above a bone into the bone, CT images are taken. After that, the processing path of robot is determined based on the CT images. At this time, the reference coordinate system of the processing path of robot is established by the pins in the CT images.

As completed the set-up of the processing path of robot, the registration is performed by matching the real pins inserted into the surgical region with the pins in the CT images, which are basis of the processing path of robot. Such the pin-basis registration method may cause pain and discomfort of patients due to pins inserted into the diseased part from start to the end of the surgery.

On the other hand, according to the image-basis registration method, the processing path of robot is determined by CT images of a thighbone of a patient, which is obtained before surgery. In the early days, the registration was made by matching three-dimensional images obtained from CT images with two-dimensional X-ray images of bones of patients obtained during surgery. Such method causes many errors in the process of distinguishing tissues like bone tissues, ligaments, etc. and the process of detecting edges. To reduce such errors, recently, the registration method that matches a particular point of a pre-surgery CT image with a particular point measured by digitizer during surgery has been being used. According to the registration method using the digitizer needs to press surface of a thighbone with a tip of measuring pin with a steady pressure in order to measure the particular point of bone tissues with the measuring pin of digitizer in surgery. When pressing the surface of a thighbone, if pressing force is too small, it causes an error in measuring the particular point, and if pressing force is too big, it causes cracks in surface of the bone. Furthermore, it causes discomfort due to many measuring points for reducing the error, and it causes difficulty for the surgeon to correspond a measuring pin exactly with a measured point guided by a monitor attached to surgery equipment.

Meanwhile, the surgeon determines a robotic surgery plan, considering three-dimensional surface data of bones obtained by computer tomography equipment (CT), etc. before surgery, and the status of patients, etc. The determined robotic surgery plan is stored in the surgery information storage unit 340 by the surgery control unit 300 according the present invention.

At this time, the robotic surgery plan applying to the present invention may be comprised of plurality of steps, and has various cutting options that are applicable to each surgery step. The surgery information storage unit 340 according to the present invention stores libraries related to such cutting options of each surgery step.

The surgery information storage unit 340 may be implemented as a form of database, and the term “database” used in the present invention means a functional element storing information, does not mean database in a strict sense like relational database, objected-oriented database. The surgery information storage unit 340 could be implemented as various forms of storage elements including a simple storage element of a form of file-base, etc.

The surgery control unit 300, accordingly, in the step that the surgeon needs additional information to modify the surgical plan during surgery, selects appropriate options in information stored in the surgery information storage unit 340 and provides the selected options.

Concretely, at the request of modifying the surgery plan of the surgeon, the surgery control unit 300 provides at least one image associated with cutting options that are applicable to corresponding surgery step. Furthermore, the surgery control unit 300 could display at least one image associated with cutting options to be superimposed on a real-time image associated with the diseased part, and could display those images to be distinguishable from one another. For example, to display the images to be distinguishable from one another, while displaying the real-time image associated to the diseased part without any processing, the surgery control unit 300 displays the image associated with cutting options by using only outlines or translucent gray scales. At this time, the surgery control unit 300 could use augmented reality technology in displaying the two images.

In addition, the surgery control unit 300 according to the present invention modifies the preset robotic surgery plan by applying the cutting options selected by the surgeon, and controls the cutting robot 100 according to the modified robotic surgery plan.

Meanwhile, the scene image obtaining unit 330 takes pictures of surgery scenes regarding diseased parts in surgery room and obtains images of surgery scenes. The preferred embodiment of the scene image obtaining unit 330 is an optical camera.

The image registration unit 310 finds the positional relation of the scene image obtaining unit 330, for example, the optical camera, and the cutting robots 100, and matches coordinates of the image of the diseased part with coordinates of the image held by the robot.

The user interface 320 displays the scene images obtained by the scene image obtaining unit 330 and displays pre-recognized position of bones, which is stored in the surgery information storage unit 340, to be superimposed on the scene images according to the control of the surgery control unit 300.

At this time, considering the relationship of matching of the scene image and the image held by the robot that is provided by the image registration unit 310, the user interface 320 displays the two images to be superimposed on each other.

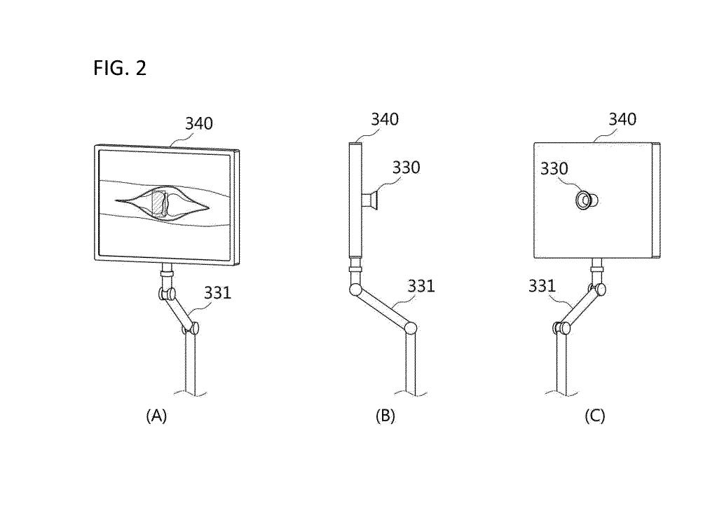

FIG. 2 shows one embodiment of the scene image obtaining unit and the user interface according to the present invention.

The embodiment of FIG. 2 illustrates an optical camera as an example of the scene image obtaining unit 330, and shows a display screen to which the optical camera is attached in the rear as an example of the user interface 340.

That is, the embodiment of FIG. 2 shows that the camera and the user interface 340 are integrated with each other. In addition, in the embodiment of FIG. 2, the camera is connected with a mechanical arm 331, and the user can move the camera 330 and the user interface 340 at the same time by moving the mechanical arm 331.

Meanwhile, in the present embodiment, a sensor included in the mechanical arm 331 can find position of the camera, and the found position of the camera is used in image registration of the image registration unit 310 according to the present invention. Furthermore, besides of the method of using the sensor included in the mechanical arm 331, the position of camera and the position of display could be found by wireless methods such as infrared rays.

In the embodiment of FIG. 2, it is easy for the user to find with naked eyes on the space because the user interface 340 locates at same position with the camera 330.

The A of FIG. 2 is a front view of a display screen to which the optical camera is attached. The B of FIG. 2 is a side view of the display screen to which the optical camera is attached. The C of FIG. 2 is a rear view of the display screen to which the optical camera is attached.

When the surgeon moves the optical camera to desired position during surgery, the computer attached to the robot displays the shape, which is to be processed by the robot, to be superimposed on the image obtained by the optical camera. It can be understood by the display screen shown in A of FIG. 2.

As moving the position of camera to desired position, the surgeon can determine whether the shape, which is to be processed by the robot, has a risk of conflicts with soft tissues. In addition, the surgeon can omit the cutting, which is possible to cause any problem, by removing a part of the shape in the surgery plan displayed superimposed by the user interface 340, or the surgeon can add the amount of cutting as he wants.

Furthermore, the apparatus for adjusting a surgery robotic plan according to the present invention provides many possible libraries of cutting paths. When the surgeon selects one of the options in the libraries, the apparatus displays the shape, which is to be processed using the selected option, to be superimposed on the real image of surgery, which is being showed currently, thereby helping a choice of the surgeon.

In addition, because the apparatus for adjusting a surgery robotic plan according to the present invention has the position of bones in advance before surgery, the apparatus can display the known position of bones to be superimposed on the real position of bones of surgery room inputted by camera, after matching those two kinds of position of bones. For example, in the position of bones previously known, when the apparatus displays outlines of bones previously known to be superimposed on the image of bones being showed currently, it can be easily understood that whether the known position of bones is correct or not.

Meanwhile, it was mentioned above that the augmented reality technology could be used when the apparatus displays the image associated with cutting options of the robotic surgery plan to be superimposed on the real image of bones showed in surgery room.

The augmented reality technology is a technology to superimpose some virtual objects on the real world that the user can see with eyes. It can be also called by mixed reality (MR), because it shows as a one image, combining the virtual world having additional information with the real world in real time. The research and development about hybrid VR system combining the real world and the virtual world have been in the progress since the late 1990s centered, especially in the United States and Japan.

In the augmented reality, which is a concept of complementing the real world with the virtual world, a leading part is the real world in spite of using the virtual world made by computer graphics. The computer graphics have a role to provide information additionally required by the real world. It means that to make ambiguous to distinguish the real world from the virtual screen by overlapping a three-dimensional virtual image on the real image showed to user.

Therefore, according to the present invention, the augmented reality technology is achieved by superimposing the image associated with cutting options of the surgery plan, which is data of the virtual world, on the image of the diseased part of the real world, which is about the target of surgery.

Meanwhile, in the present embodiment, the apparatus for adjusting a robotic surgery plan according to the present invention adjusts the camera 330 toward the robot or sensors attached to the robot, and displays outlines of robot, which is previously known, to be superimposed on the real image of the robot inputted by the camera. Accordingly, it can be easily understood that whether the relationship of measured position between the robot and camera is correct.

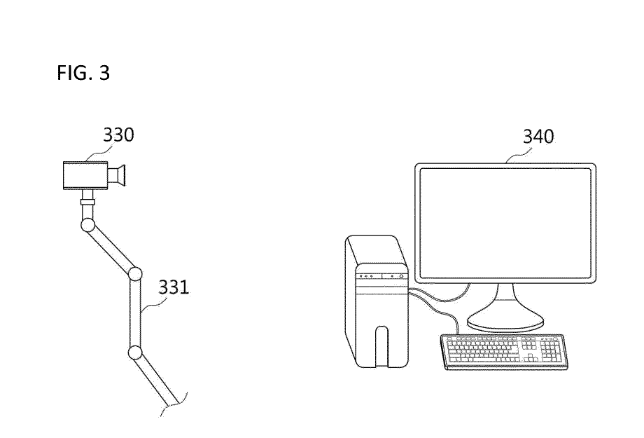

FIG. 3 shows an anther embodiment of the scene image obtaining unit and the user interface.

The embodiment of FIG. 3, as an example of the scene image obtaining unit 330, also illustrates the optical camera that is attached to the mechanical arm 331 to move with the mechanical arm 331. The different thing with the embodiment of FIG. 2 is that the user interface 340 is not attached with the optical camera, but locates away from the optical camera to give user comfort to see.

The optical camera 330 and the user interface 340 could communicate with each other by wired or wireless network.

In this case, as in the other case, the sensor of the mechanical arm 331 can find position of the camera. The found position of the camera is used in image registration of the image registration unit 310. The position of the camera also could be found by using wireless methods like infrared rays, etc. besides using the sensor attached to the mechanical arm 331.

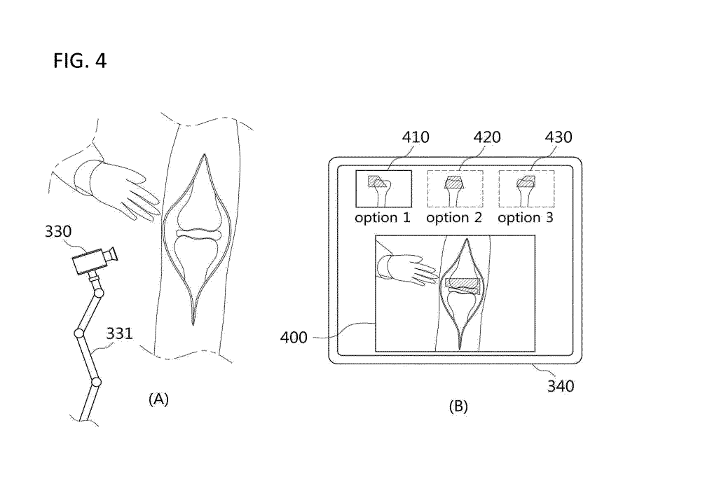

FIG. 4 shows an example of a screen of cutting options provided during surgery by the apparatus for adjusting a robotic surgery plan according to the present invention.

As illustrated in A of FIG. 4, during surgery, when the surgeon moves the optical camera 330 to desired position, in other words, the surgeon moves the optical camera 330 to above an exposed bone, the apparatus displays the image that is to be processed by the robot to be superimposed on the image obtained by the optical camera via the user interface 340.

As referring B of FIG. 4, in the situation that the predetermined surgery plan has been suspended, the apparatus displays alternative cutting options 410, 420, 430 on top of the main screen 400 of the user interface 340.

The cutting options are displayed to be superimposed on the image of the exposed bone. When the surgeon selects one option among those cutting options, the main screen 400 displays the selected cutting option to be superimposed on the real image of the diseased part. The image on the main screen 400 of FIG. 4 is a related image in the case that the user selected option 1 among three options.

At this time, the surgery information storage unit stores libraries of possible cutting path, and the apparatus according to the present invention provides the libraries of possible cutting path to the user, thereby helping the user make choices.

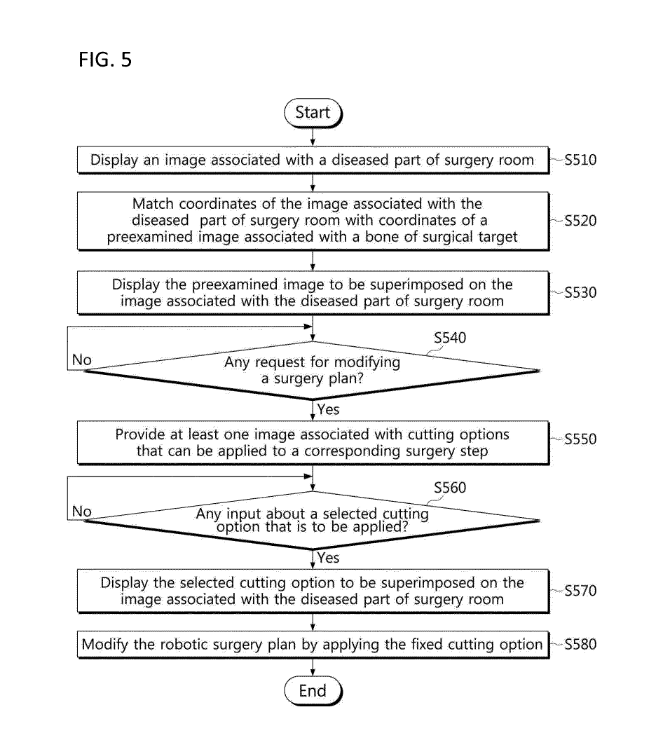

FIG. 5 is a flowchart that depicts the method for adjusting a robotic surgery plan according to the present invention.

In explanation about an embodiment hereinafter, although it can be understood that each step of the method for adjusting a robotic surgery plan according to the present invention is performed in corresponding elements of the apparatus for adjusting a robotic surgery plan, which was explained through FIG. 1, the each step of the method should be limited as function itself, which defines the each step. In other words, the performer of each step is not limited by the names of elements that are given as examples of performer of each step.

According to the method for adjusting a robotic surgery plan, in step S510, an image associated with a diseased part of surgery room, which is obtained by the optical camera, etc. is displayed. When the image associated with the diseased part of surgery room is obtained, in step S520, the apparatus matches coordinates of the image associated with the diseased part with coordinates of an image associated with a bone of surgical target that has been already obtained by equipment like CT, etc. As the matching is complete, in step S530, the apparatus displays the pre-examined image associated with the bone of surgical target to be superimposed on the real-time image associated with the diseased part of surgery room.

After that, when receiving a request for modifying a surgery plan from a surgeon in step S540, the apparatus provides at least one image associated with cutting options, which can be applied to a corresponding surgery step in step S550. In step S560, when a selected cutting option that is to be applied is inputted, in step S570, the apparatus displays the selected cutting option to be superimposed on the image associated with the diseased part of surgery room. In step S580, as the selected cutting option is fixed, the apparatus modifies the surgery plan, applying the fixed cutting option.

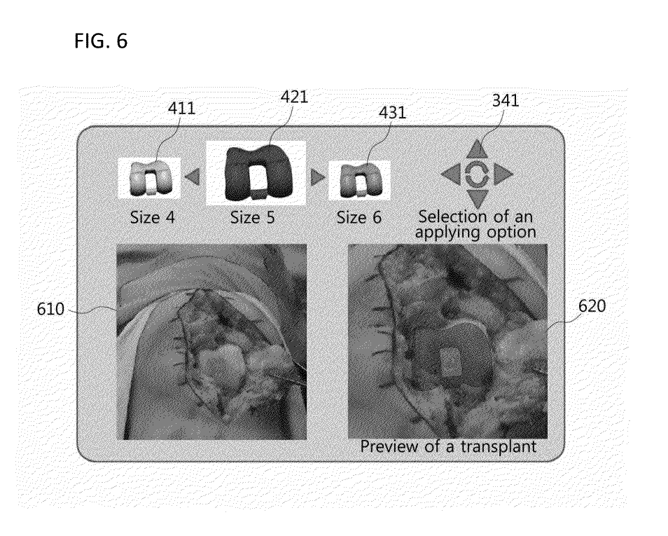

FIG. 6 illustrates an example of a screen of the user interface in which the present invention can be applied.

The screen of the user interface of FIG. 6 shows an example of a screen providing the various processing options that can be applied to a bone transplant surgery that cuts real bones and transplants artificial bones, and displaying the selected option superimposed on the real image of bones.

In FIG. 6, the images associated with processing options 411, 421, 431 according to the present invention are displayed on top of the screen. Furthermore, in FIG. 6, on the top right-hand side of the screen of the user display device, a menu for selecting options 341 is provided, so that the surgeon can select a processing option that is to be applied to the robotic surgery.

In FIG. 6, on the bottom left-hand side of the screen 610 of user display device, an image of a real diseased part is displayed, and on the bottom right-hand side 620, an image of the selected processing option is displayed in addition to the image of the real diseased part. In other words, on the bottom right-hand side of the screen 620, the processing option of selected size 5 is displayed to be superimposed on the real image of the diseased part using augmented reality, thereby providing the surgeon with predicted appearances of the diseased part when a bone transplant surgery has been performed by applying the processing option of size 5. If the surgeon thinks that the transplant model of size 5 is not matched with the status of real bone of diseased part properly, the surgeon can select a processing option of the most proper size by selecting other option.

According to the present invention, after advance checking a virtual preview of transplant of when the provided processing options is applied to the real diseased part, the surgeon modifies the surgery plan by selecting and determining the processing option of the most proper size. Accordingly, the surgeon can adjust the robotic surgery so that the robotic surgery is performed by the modified surgery plan.

According to the present invention that has been described above with the embodiments, the present invention can deal with various requests for modifying robotic surgery plans actively and promptly.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. An apparatus for adjusting a robotic surgery plan, comprising:
a surgery information storage unit storing an examined first image associated with an inputted robotic surgery plan and a target bone of surgery;
a scene image obtaining unit obtaining a second image associated with a diseased part in real time in surgery room;
an image registration unit matching coordinates of the examined first image with coordinates of the second image associated with the diseased part;
a user interface displaying the examined first image and the second image associated with the diseased part; and
a surgery control unit controlling the user interface to display the examined first image to be superimposed on the second image associated with the diseased part, which is inputted in real time.

2. The apparatus according to claim 1, wherein the surgery information storage unit further stores phased cutting options of the robotic surgery plan and related images thereof.

3. The apparatus according to claim 2, wherein the surgery control unit provides at least one image associated with the cutting options that is applicable to a corresponding surgery step, according to request for modifying the surgery plan inputted via the user interface.

4. The apparatus according to claim 3, wherein the surgery control unit controls the user interface so that at least one image associated with the cutting options is superimposed on the second image associated with the diseased part, and also displayed to be distinguishable from the second image associated with the diseased part.

5. The apparatus according to claim 1, wherein the scene image obtaining unit comprises an optical camera and a mechanical arm which is attached to the optical camera and supports movements of the optical camera.

6. The apparatus according to claim 1, wherein the scene image obtaining unit and the user interface are attached to each other to be moveable together.

7. The apparatus according to claim 1, wherein the surgery control unit controls the user interface so that the user interface displays outlines of the first image to be superimposed on the second image associated with the diseased part.

8. The apparatus according to claim 7, wherein the surgery control unit displays the outlines of the first image to be superimposed on the second image associated with the diseased part using augmented reality technology.

9. The apparatus according to claim 3, wherein the surgery control unit modifies the robotic surgery plan based on a selected cutting option.

10. The apparatus according to claim 9, further comprising a cutting robot processing a target bone of surgery according to the modified robotic surgery plan inputted from the surgery control unit.
